(19) United States
(12) Patent Application Publication
(10) Pub. No.: US 2006/0056671 A1
(43) Pub. Date: Mar. 16, 2006

(54) Automated feature extraction processes and systems

(76) Inventors: Jayati Ghosh, San Jose, CA (US); Christian A. Le Cocq., Menlo Park, CA (US); Xiangyang Zhou, Mountain View, CA (US); John Frederick Corson, Mountain View, CA (US); Glenda Choate Delenstarr, Redwood City, CA (US); Charles David Troup, Livermore, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/941,501

(22) Filed: Sep. 15, 2004

(51) Int. Cl. G06K 9/00; G06K 9/46; C12M 1/34

(52) U.S. Cl. 382/128; 382/190; 435/288.4

(57) ABSTRACT

Methods, systems and computer readable media for automatically feature extracting array images in batch mode. At least two images to be feature extracted are loaded into a batch project, and the system automatically and sequentially feature extracts the images in the batch project. At least one of the images may be feature extracted based upon a different grid template or protocol than at least one other of the images. Methods, systems and computer readable media are provided to automatically feature extract a single array image having an identifier indicating that it is a multipack, multiple array image. A system provided for feature extracting array images in batch mode includes a user interface with a feature enabling a user to select images to be loaded into a batch project; and based upon the loaded images in the batch project, the system may automatically assign a grid template to each image loaded into the batch project. Further, the system may automatically assign a protocol to each image loaded into the batch project.

### BACKGROUND OF THE INVENTION

Array assays between surface bound binding agents or probes and target molecules in solution are used to detect the presence of particular biopolymers. The surface-bound probes may be oligonucleotides, peptides, polypeptides, proteins, antibodies or other molecules capable of binding with target molecules in solution. Such binding interactions are the basis for many of the methods and devices used in a variety of different fields, e.g., genomics (in sequencing by hybridization, SNP detection, differential gene expression analysis, comparative genomic hybridization, identification of novel genes, gene mapping, finger printing, etc.) and proteomics.

One typical array assay method involves biopolymeric probes immobilized in an array on a substrate such as a glass substrate or the like. A solution containing analytes that bind with the attached probes is placed in contact with the array substrate, covered with another substrate such as a coverslip or the like to form an assay area and placed in an environmentally controlled chamber such as an incubator or the like. Usually, the targets in the solution bind to the complementary probes on the substrate to form a binding complex. The pattern of binding by target molecules to biopolymer probe features or spots on the substrate produces a pattern on the surface of the substrate and provides desired information about the sample. In most instances, the target molecules are labeled with a detectable tag such as a fluorescent tag or chemiluminescent tag. The resultant binding interaction or complexes of binding pairs are then detected and read or interrogated, for example by optical means, although other methods may also be used. For example, laser light may be used to excite fluorescent tags, generating a signal only in those spots on the biochip (substrate) that have a target molecule and thus a fluorescent tag bound to a probe molecule. This pattern may then be digitally scanned for computer analysis.

As such, optical scanners play an important role in many array based applications. Optical scanners act like a large field fluorescence microscope in which the fluorescent pattern caused by binding of labeled molecules on the array surface is scanned. In this way, a laser induced fluorescence scanner provides for analyzing large numbers of different target molecules of interest, e.g., genes/mutations/alleles, in a biological sample.

Scanning equipment used for the evaluation of arrays typically includes a scanning fluorometer. A number of different types of such devices are commercially available from different sources, such as Perkin-Elmer, Agilent Technologies, Inc., Axon Instruments, and others. In such devices, a laser light source generates a collimated beam. The collimated beam is focused on the array and sequentially illuminates small surface regions of know location on an array substrate. The resulting fluorescence signals from the surface regions are collected either confocally (employing the same lens to focus the laser light onto the array) or off-axis (using a separate lens positioned to one side of the lens used to focus the laser onto the array). The collected signals are then transmitted through appropriate spectral filters, to an optical detector. A recording device, such as a computer memory, records the detected signals and builds up a raster scan file of intensities as a function of position, or time as it relates to the position.

Analysis of the data (the stored file) may involve collection, reconstruction of the image, feature extraction from the image and quantification of the features extracted for use in comparison and interpretation of the data. Where large numbers of array files are to be analyzed, the various arrays from which the files were generated upon scanning may vary from each other with respect to a number of different characteristics, including the types of probes used (e.g., polypeptide or nucleic acid), the number of probes (features) deposited, the size, shape, density and position of the array of probes on the substrate, the geometry of the array, whether or not multiple arrays or subarrays are included on a single slide and thus in a single, stored file resultant from a scan of that slide, etc.

Processing of multiple files to date, has involved a substantial amount of user interaction and time-consuming set up and user input in order to process the files. For example, the user may be prompted provide input to a computer processor to aid in locating corners, features and/or other array characteristics on a displayed image of the array signal data of a stored file. When feature extraction processing is completed, the next stored file is then loaded to repeat the process, and this again requires user interaction as described. Given that an array may contain thousands or hundreds of thousands of features and that each feature may result in ten, twenty or more pixels of array signal data, feature extraction and analysis as described can be time consuming and require a high degree of operator input, at least intermittently, throughout the process. Thus, high throughput reading and feature extraction of arrays is not efficiently achieved by these techniques.

GenePix® Pro 6.0 from Molecular Devices Corporation (Axon Instruments) http://www.axon.com/GN_GenePixSoftware.html provides microarray image analysis software that includes very restrictive batch processing capabilities. The batch analysis mode of this software is designed for a very specific automation task, to analyze all images from a batch that use the same setting file or GAL file. The images analyzed must be multi-image TIFF files, or, if single image, must be in named pairs. The images must be analyzable using the same settings or GAL file, without human intervention to tweak block and feature-indicator positions. Although this software provides an efficiency advantage for a very restrictive subset of all batch processing, the batch analysis features are not useful for any other types/configurations of images. Even for batches that can be processed with the batch analysis feature, the software does not allow user intervention where and when it is needed.

ImaGene from BioDiscovery http://www.biodiscovery.com/imagene.asp provide microarray image analysis software that it is believed may offer some limited form of batch processing capabilities. Using this batch mode feature, it is believed that a first image must first be set up and “run” (e.g., to do feature extraction) and then a grid template and configuration file for the run are saved. It is believed that a plurality of images may then be loaded and run according to the saved characteristics of the grid template and configuration file. This feature is quite restrictive in that all images must be processed according to the same grid template and configuration file. Also, it is not known whether any user intervention or input is allowed, once a batch process of this type has been set up and/or initiated.

There remain continuing needs for improved solutions for efficiently analyzing scanned array images to reduce user input requirements, thereby reducing the costs of processing and potentially increasing the throughput speed of such analysis. Further, reliability of results would be improved by reducing incidence of human input error. Such needs are especially strong felt for batch processing of images that are not all of uniform configuration, protocol, etc. At the same time, it would be desirable to maintain flexibility so that a user has an option of inputting information or overriding automated features when desired.

### SUMMARY OF THE INVENTION

Embodiments of the present invention include methods, systems and computer readable media for automatically feature extracting array images in batch mode. At least two at least two images to be feature extracted are loaded into a batch project, and the images are automatically and sequentially feature extracted. At least one of the images may be feature extracted based upon a different grid template or protocol than at least one other of the images.

Methods, systems and computer readable media are also provided for automatically feature extracting a single array image having an identifier indicating that it is a multipack, multiple array image. An attempt is made to overlay an assigned grid template over multiple spaces considered to be occupied by multiple arrays on the image as indicated by a design file upon which the grid template is based. The dimensions of the attempted overlays are then compared to the dimensions of the image. If at least one of the dimensions of the overlays is larger than the corresponding dimension of the image (i.e., not all of attempted overlays will fit inside the scan dimensions of the image), then it is determined that the image is a single array image, and the grid template is overlaid only once over the image to locate the features in the single array.

An embodiment of a system for feature extracting array images in batch mode includes a user interface with a feature enabling a user to select images to be loaded into a feature extraction project; means for automatically assigning a grid template to each image loaded into the feature extraction project; and means for automatically assigning a protocol to each image loaded into the feature extraction project.

The present invention also covers forwarding, transmitting and/or receiving results from any of the methods described herein.

These and other advantages and features of the invention will become apparent to those persons skilled in the art upon reading the details of the methods, systems and computer readable media as more fully described below.

### BRIEF DESCRIPTION OF THE DRAWINGS

FIG. 1 is a representation of information that may be included in a design file for a grid template.

FIG. 2 is a simple illustration of a scanned image, in which the image has two arrays or subarrays each having three rows and four columns of features.

FIG. 3A is a simple illustration of a scanned image having a dense-packed array.

FIGS. 3B-3C are illustrations demonstrating two different conventions for mapping the features of a dense pack array.

FIG. 4 shows a portion of a feature extraction project tree.

FIGS. 5A-5B show a scanned image and a grid template useful for locating the features of the arrays on the scanned image.

FIGS. 5C-5D show a scanned image having arrays with a systematic error and a grid template modified so as to be useful for locating the features of the arrays exhibiting the systematic error.

FIG. 6 is a simple illustration representing an eight pack scanned image, including one array that was a “mock hyb”.

FIG. 7 is an illustration of a screen view that provides a user with a feature extraction project and tree and various utilities for setting up, running and directing resulting outputs.

FIG. 8 illustrates a typical computer system that may be used to practice an embodiment of the present invention.

### DETAILED DESCRIPTION OF THE INVENTION

Before the present systems, methods and computer readable media are described, it is to be understood that this invention is not limited to particular software, hardware, process steps or substrates described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a slide” includes a plurality of such slides and reference to “the array” includes reference to one or more arrays and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

A “microarray”, “bioarray” or “array”, unless a contrary intention appears, includes any one-, two-or three-dimensional arrangement of addressable regions bearing a particular chemical moiety or moieties associated with that region. A microarray is “addressable” in that it has multiple regions of moieties such that a region at a particular predetermined location on the microarray will detect a particular target or class of targets (although a feature may incidentally detect non-targets of that feature). Array features are typically, but need not be, separated by intervening spaces. In the case of an array, the “target” will be referenced as a moiety in a mobile phase, to be detected by probes, which are bound to the substrate at the various regions. However, either of the “target” or “target probes” may be the one, which is to be evaluated by the other.

Methods to fabricate arrays are described in detail in U.S. Pat. Nos. 6,242,266; 6,232,072; 6,180,351; 6,171,797 and 6,323,043. As already mentioned, these references are incorporated herein by reference. Other drop deposition methods can be used for fabrication, as previously described herein. Also, instead of drop deposition methods, photolithographic array fabrication methods may be used. Interfeature areas need not be present particularly when the arrays are made by photolithographic methods as described in those patents.

Following receipt by a user, an array will typically be exposed to a sample and then read. Reading of an array may be accomplished by illuminating the array and reading the location and intensity of resulting fluorescence at multiple regions on each feature of the array. For example, a scanner may be used for this purpose is the AGILENT MICROARRAY SCANNER manufactured by Agilent Technologies, Palo, Alto, Calif. or other similar scanner. Other suitable apparatus and methods are described in U.S. Pat. Nos. 6,518,556; 6,486,457; 6,406,849; 6,371,370; 6,355,921; 6,320,196; 6,251,685 and 6,222,664. Scanning typically produces a scanned image of the array which may be directly inputted to a feature extraction system for direct processing and/or saved in a computer storage device for subsequent processing. However, arrays may be read by any other methods or apparatus than the foregoing, other reading methods including other optical techniques or electrical techniques (where each feature is provided with an electrode to detect bonding at that feature in a manner disclosed in U.S. Pat. Nos. 6,251,685, 6,221,583 and elsewhere).

A “design file” is typically provided by an array manufacturer and is a file that embodies all the information that the array designer from the array manufacturer considered to be pertinent to array interpretation. For example, Agilent Technologies supplies its array users with a design file written in the XML language that describes the geometry as well as the biological content of a particular array.

A “grid template” or “design pattern” is a description of relative placement of features, with annotation, that has not been placed on a specific image. A grid template or design pattern can be generated from parsing a design file and can be saved/stored on a computer storage device. A grid template has basic grid information from the design file that it was generated from, which information may include, for example, the number of rows in the array from which the grid template was generated, the number of columns in the array from which the grid template was generated, column spacings, subgrid row and column numbers, if applicable, spacings between subgrids, number of arrays/hybridizations on a slide, etc. An alternative way of creating a grid template is by using an interactive grid mode provided by the system, which also provides the ability to add further information, for example, such as subgrid relative spacings, rotation and skew information, etc.

A “grid file” contains even more information than a “grid template”, and is individualized to a particular image or group of images. A grid file can be more useful than a grid template in the context of images with feature locations that are not characterized sufficiently by a more general grid template description. A grid file may be automatically generated by placing a grid template on the corresponding image, and/or with manual input/assistance from a user. One main difference between a grid template and a grid file is that the grid file specifies an absolute origin of a main grid and rotation and skew information characterizing the same. The information provided by these additional specifications can be useful for a group of slides that have been similarly printed with at least one characteristic that is out of the ordinary or not normal, for example. In comparison when a grid template is placed or overlaid on a particular microarray image, a placing algorithm of the system finds the origin of the main grid of the image and also its rotation and skew. A grid file may contain subgrid relative positions and their rotations and skews. The grid file may even contain the individual spot centroids and even spot/feature sizes.

A “history” or “project history” file is a file that specifies all the settings used for a project that has been run, e.g., extraction names, images, grid templates protocols, etc. The history file may be automatically saved by the system and is not modifiable. The history file can be employed by a user to easily track the settings of a previous batch run, and to run the same project again, if desired, or to start with the project settings and modify them somewhat through user input.

“Image processing” refers to processing of an electronic image file representing a slide containing at least one array, which is typically, but not necessarily in TIFF format, wherein processing is carried out to find a grid that fits the features of the array, to fine individual spot/feature centroids, spot/feature radii, etc. Image processing may even include processing signals from the located features to determine mean or median signals from each feature and may further include associated statistical processing. At the end of an image processing step, a user has all the information that can be gathered from the image.

“Post processing” or “post processing/data analysis”, sometimes just referred to as “data analysis” refers to processing signals from the located features, obtained from the image processing, to extract more information about each feature. Post processing may include but is not limited to various background level subtraction algorithms, dye normalization processing, finding ratios, and other processes known in the art.

A “protocol” provides feature extraction parameters for algorithms (which may include image processing algorithms and/or post processing algorithms to be performed at a later stage or even by a different application) for carrying out feature extraction and interpretation from an image that the protocol is associated with. Protocols are user definable and may be saved/stored on a computer storage device, thus providing users flexibility in regard to assigning/pre-assigning protocols to specific microarrays and/or to specific types of microarrays. The system may use protocols provided by a manufacturer(s) for extracting arrays prepared according to recommended practices, as well as user-definable and savable protocols to process a single microarray or to process multiple microarrays on a global basis, leading to reduced user error. The system may maintain a plurality of protocols (in a database or other computer storage facility or device) that describe and parameterize different processes that the system may perform. The system also allows users to import and/or export a protocol to or from its database or other designated storage area.

An “extraction” refers to a unit containing information needed to perform feature extraction on a scanned image that includes one or more arrays in the image. An extraction includes an image file and, associated therewith, a grid template or grid file and a protocol.

A “feature extraction project” or “project” refers to a smart container that includes one or more extractions that may be processed automatically, one-by-one, in a batch. An extraction is the unit of work operated on by the batch processor. Each extraction includes the information that the system needs to process the slide (scanned image) associated with that extraction.

When one item is indicated as being “remote” from another, this is referenced that the two items are at least in different buildings, and may be at least one mile, ten miles, or at least one hundred miles apart.

“Communicating” information references transmitting the data representing that information as electrical signals over a suitable communication channel (for example, a private or public network).

“Forwarding” an item refers to any means of getting that item from one location to the next, whether by physically transporting that item or otherwise (where that is possible) and includes, at least in the case of data, physically transporting a medium carrying the data or communicating the data.

A “processor” references any hardware and/or software combination which will perform the functions required of it. For example, any processor herein may be a programmable digital microprocessor such as available in the form of a mainframe, server, or personal computer. Where the processor is programmable, suitable programming can be communicated from a remote location to the processor, or previously saved in a computer program product. For example, a magnetic or optical disk may carry the programming, and can be read by a suitable disk reader communicating with each processor at its corresponding station.

Reference to a singular item, includes the possibility that there are plural of the same items present.

“May” means optionally.

Methods recited herein may be carried out in any order of the recited events which is logically possible, as well as the recited order of events.

All patents and other references cited in this application, are incorporated into this application by reference except insofar as they may conflict with those of the present application (in which case the present application prevails).

In order to perform feature extraction, the system requires three components for each extraction performed. One component is the image (scan) itself, which may be a file saved in an electronic storage device (such as a hard drive, disk or other computer readable medium readable by a computer processor, for example). Typically, the image file is in TIFF format, as this is fairly standard in the industry, although the present invention is not limited to use only with TIFF format images. The second component is a grid template or design file (although a grid file may be substituted as will be discussed further below) that maps out the locations of the features on the array from which the image was scanned and indicates which genes or other entities that each feature codes for.

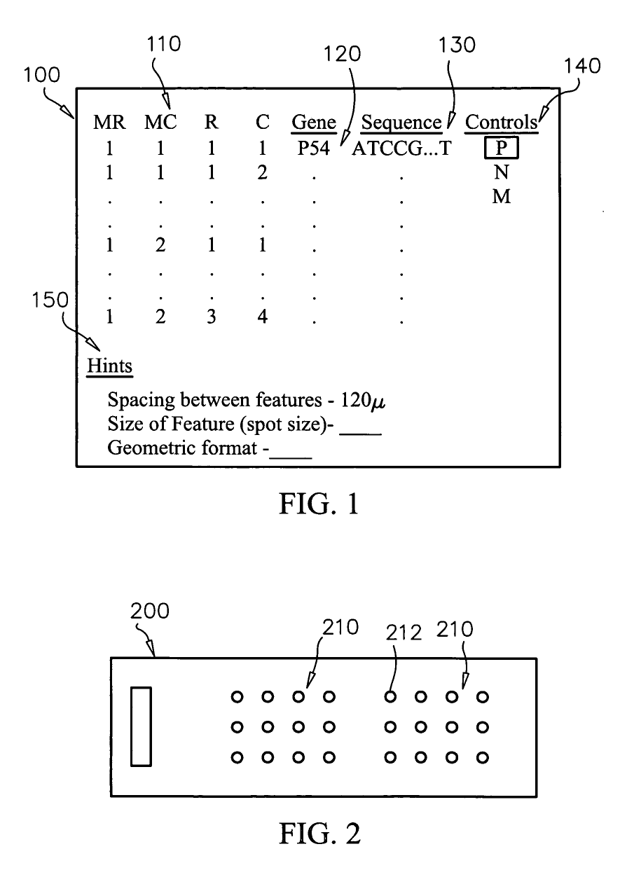

FIG. 1 is a representation of information that may be included in a design file 100 for a grid template. In this example, the feature coordinates 110 are listed for a slide or scanned image 200 having two subarrays 210 each having three rows and four columns, see FIG. 2. For each feature on the image, feature coordinates 110 may be provided in grid template 100. Each feature may be identified by the row and column in which it appears, as well as meta-row and meta-column, that identify which array or subarray that the feature appears in when there are multiple arrays/subarrays on a single slide 100. Thus, for example, the coordinates that read 1 2 1 1 in FIG. 1 refer to feature 212 shown in FIG. 2, that is in row 1, column 1 of the subarray located in meta-row 1, meta-column 2. Note that there is only one row of subarrays (i.e., one meta-row) and two columns of subarrays (i.e., two meta-columns).

For each feature, the gene or other entity 120 that that feature codes for may be identified adjacent the feature coordinates. Also, the specific sequence 130 (e.g., oligonucleotide sequence or other sequence) that was laid down on that particular feature may also be identified relative to the mapping information/feature coordinates. Controls 140 used for the particular image may also be identified. In the example shown in FIG. 1, positive controls are used. Typical control indications include, but are not limited to, positive, negative and mismatched. Positive controls result in bright signals by design, while negative controls result in dim signals by design. Mismatched or deletion control provides a control for every probe on the array.

“Hints” 150 may be provided to further characterize an image to be associated with a grid template 100. Hints may include: interfeature spacing (e.g., center-to-center distance between adjacent features), such as indicated by the value 120μ in FIG. 1; the size of the features appearing on the image (e.g., spot size); the geometric format of the array or arrays (e.g., rectangular, dense pack, etc.), spacing between subarrays, etc. The geometric format may be indicated as a hint in the same style that the individual features are mapped in 110. Thus, for example, a hint as to the geometric format of slide 200 may indicate rectangular, 1 2 3 4. Hints assist the system in correctly placing the grid template 100 on the grid formed by the feature placement on a slide/image.

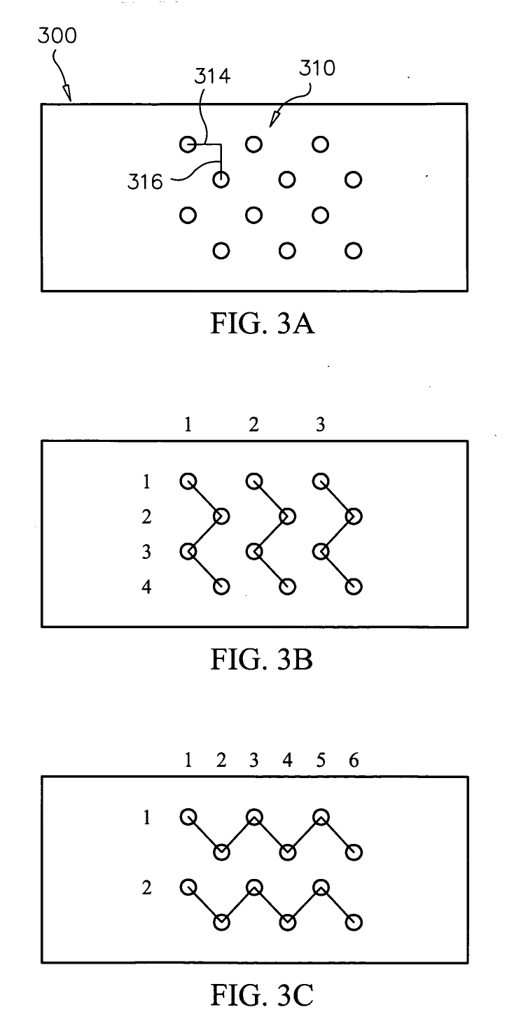

FIG. 3A shows a representation of an image/slide arranged with dense pack geometry 310. for this type of configuration, hints 150 may include center to center offset distances 314, 316 between features 312 in the X and Y directions. Column identification and row identification for this type of configuration may be addressed in one of two different ways. One is by considering the columns to “zig-zag” as assumed by the rows and columns identified in FIG. 3B, where zig-zagged columns are shown connected by lines, and the other is by considering the rows to “zig-zag” as assumed by the rows and columns identified in FIG. 3C, where zig-zagged rows are shown connected by lines. Note that use of the two different conventions give different results, as FIG. 3B is considered to have four rows and three columns, while the very same array, when counted according to the convention in FIG. 3C gives two rows and six columns. Although the system defaults to the convention shown in FIG. 3B, the present invention is not limited to this invention, as it could just as well be practiced using the convention shown in FIG. 3C.

The third component required for an extraction is a protocol. The protocol defines the processes that the system will perform on the image file that it is associated with. Examples of processes that may be identified in the protocol to be carried out on the image file include, but are not limited to: local background subtraction, negative control background subtraction, dye normalization, selection of a specific set of genes to be used as a dye normalization set upon which to perform dye normalization, etc. The system may include a database in which grid templates and protocols may be stored for later call up and association with image files to be processed. The system allows a user to create and manage a list of protocols, as well as a list of grid templates.

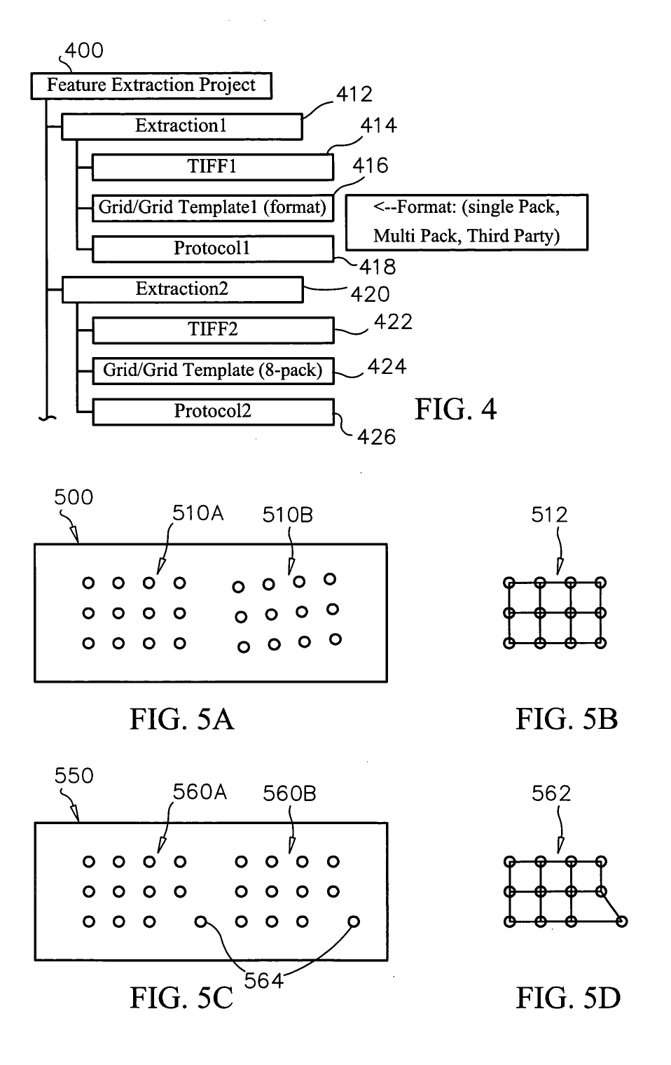

In one embodiment, a feature extraction project may be set up to have grid templates and/or protocols to image files by default. In use, a user may open a Feature Extraction Project by clicking on a Feature Extraction Project Node 400 (FIG. 4) on the system display. Of course, it is to be understood that the presentation of the Feature Extraction Project to a user does not have to employ a tree and node architecture, as other structures could be alternatively employed to carried out the same functions and processes, as would be readily apparent to those of ordinary skill in the art. The Feature Extraction Project Node 400 may initially be empty. Once opened, the user can than access his/her image files and add one or more of these files into the open project tree 410. An image can be added to a project in multiple different ways, one of which is when the user accesses a computer folder or directory and drags and drops the image file from the folder or directory into the project tree 410. Upon adding the image file, an extraction node is automatically created in project tree 410, see Extraction1 412 for this example.

As it is automatically created, the extraction unit (e.g., extraction node or other representative construct that is used to contain the image file, grid template and protocol, and may be automatically created) is automatically named with the name of the image (TIFF) file, unless that extraction name already exists in the project tree 410. In such instances, the system changes the name to include the name of the image file in addition to a suffix being placed at the end of the name. For example, if the name of the image file Tiff1 414 being dragged to Exraction1 is “US 12302345_{—}16012064010028_S01”, then node Extraction1 412 is automatically named US12302345_{—}16012064010028_S01 by the system. However, if the user then adds the same file to project tree 410 again, for example to be processed according to another protocol, or for some other reason, then a second extraction node Extraction2 is automatically named by the system as US12302345_{—}16012064010028_S01-2. Each extraction name is modifiable and contains free text. Duplicate names to previous extractions cannot be made.

In addition to automatically creating an extraction unit (i.e., an extraction) for each image file added to the project, the system may also automatically associate a grid template and/or a protocol with each image under each extraction unit. There are at least two ways that a grid template can be automatically associated with an image file that has been assigned to an extraction unit. The system may provide a database in which available grid templates and protocols may be stored. For example, all of the protocols that are typically used by a given laboratory may be stored in the database for users that work in that laboratory.

Scanned slides/arrays often, but not always include a barcode or other identifier, which is scanned at the same time that the array or arrays on the slide are scanned. The barcode or identifier information may be stored in the scanned image file. In this instance, when the image file is added to the project, the system reads the associated text from the barcode/identifier information. This information (or a portion thereof, sometimes referred to as an array ID) may also be linked to a particular grid file that characterizes the image file, and if it does, the system automatically populates the extraction that the image is assigned to with that grid file. For example, the barcode (or design ID portion thereof) associated with image TIFF1 414 was linked with Grid Template1, which the system automatically added to Extraction1 412.

If an image file being added to the project does not have a barcode or similar identifier associated with it, then the system cannot read specific information for linking with a particular grid template. In this instance, the system populates the extraction with a default grid template for this project. A default grid template may be a grid template that is typically used by the laboratory running the project for example. Another possible project level option is “use grid file”, which may be a more individualized type of format, as will be discussed in greater detail below.

It should be noted that a user of the system has the ability to view the automatic population of the project tree 410 or other graphic representation of the Feature Extraction project by the system in response to the user's input of image files to be processed. Additionally, the user also is afforded the option of changing any of the automatically populated parameters, should the user decide to do so. Thus, for example, if a user knows that a particular default grid is not the appropriate grid to use for an image file added, but that the default grid is assigned to this image because there is no barcode or identifier associated with the image, the user can go into the display of the project and change the grid template to a better suited one to be associated with that particular image.

Referring back to an earlier example, if the user adds the same image file twice to the same Feature Extraction Project 400/project tree 410b or other graphical representation of a project displayed on a user interface (e.g., the same image is added as TIFF1 414 and TIFF2 422) then the system will automatically populate both Extrraction1 and Extraction2 with the same protocol. However, the user may want to process the image under different protocol conditions. In this instance, the user went into Extraction1 and changed the protocol from Protocol1 to Protocol2.

Each grid template that is maintained in the database may have a default protocol associated with it. When an image file is added to an extraction and the image file has a barcode or other identifier that the system can use to identify a linked grid template, that grid template is automatically populated with the image file in the extraction, as already noted above. Additionally, the system identifies the default protocol that is associated with the grid template that was automatically populated, and automatically populates that default protocol in the extraction along with the image file and automatically populated grid file.

In cases where a default grid template is automatically populated, as described above, the default grid template also has a default protocol associated with it, and the system identifies that default protocol and automatically populates it in the extraction along with the image and the default grid template.

An important advantage of the present system and the manner in which it processes, is that the images populated in the extractions to be processed in a batch process (Feature Extraction Process) may be processed according to different protocols, and they may also have different grid configurations. Once the Feature Extraction Project is appropriately configured, which may occur automatically upon adding image files into a Feature Extraction Project as described above, then the system can automatically process the images as a batch process, one at a time, without further human intervention.

In cases where a grid template to be populated in an extraction does not have a default protocol associated with it, the system will then look to the Feature Extraction Project/container 400 to determine whether a default protocol is associated with the project. If the project has a default protocol associated with it, then the system populates the current extraction with the default protocol. If the project does not have a default protocol associated with it, then the protocol for the current extraction is left unpopulated, and the user will need to manually assign a protocol before the current extraction can be processed. Similarly, if a default grid template cannot be identified and automatically populated, then the grid template for that extraction is left unpopulated, and the user will need to manually populate the grid template for that extraction before that extraction can be processed. For the batch processing to proceed, each extraction needs to be populated with an image file, grid template and protocol.

The system automates the batch setups as much as possible, which is beneficial to users who run the same or similar processes every day. On the other hand, the system maintains flexibility by allowing a user to override any of the automatically populated information and change it to manually inputted information that the user prefers.

Protocols that are run within a batch (feature extraction process) do not have to be the same, and this is a great advantage over earlier solutions. For example, one protocol might be a two-color experiment, a second image may be run with a protocol for test parameters with no dye normalization, and a third image may be processed according to a protocol for a one color experiment, etc. The point is that each image processing is not limited to any particular protocol and each one can even be different.

Each grid template that is stored in a database by the system identifies at least a basic geometry of an image that it will be associated with. That geometry has a certain rigidity or regularity, so that the grid template can be defined to the extent where it can be overlaid on an image to locate the grid defined by the image. However, the actual grid or array that has been deposited on a slide may be slightly skewed or rotated with respect to the slide, resulting in a similarly skewed or rotated scanned image. The system applies software techniques when overlaying the grid template to match a corner or corners of the image with the grid template, based on hints in the design file for the grid template, and to adjust for skew and/or rotation. Exemplary techniques for this part of the processing are disclosed in co-pending, commonly assigned application Serial No. 10,449,175 filed May 30, 2003 and titled “Feature Extraction Methods and Systems”. Application Serial No. 10,449,175 is hereby incorporated by reference in its entirety. FIG. 5B shows a schematic representation of a template 512 that was constructed to fit arrays having the geometry shown by subarrays 510A and 510B in FIG. 5A. Note that using the techniques as described above, template 512 may be overlaid to locate not only the features of subarray 510A, but also the features of subarray 510B, even through subarray 510B is rotated with respect to the X and Y axes (i.e., horizontal and vertical axes of the slide/image 500).

The system may provide a metric for identifying and defining to a user, how well a particular grid template/grid fits the features contained on an image. Thus, for example, after fitting the grid to the image as described above, the system may determine how far each spot or feature on the grid/grid template had to be moved in order to overlay the features on the image. These statitistics may be outputted to the user and/or a warning may be outputted to the user interface on a per extraction basis, for those extractions where the grid template was considered by the system to not be a good fit with the image that it was overlaid on, thus bringing to the user's attention that they may want to look at a particular extraction in greater detail after the automatic processing of the batch has completed. For example, a predetermined threshold for determining a poor fit may be when fifty percent or more of all spots had to be moved by a distance greater than or equal to the nominal diameter of the spots, in order to overlay the features on the image. However, such threshold may be modified, for example the distance may be set to greater than or equal to three quarters of the nominal diameter, or half the nominal diameter. Further, the threshold cutoff may be reached when less than fifty percent of the spots qualify by being moved by at least the threshold distance.

There may be arrays that have anomalies or abnormalities that make it impossible for a grid template to be accurately fitted on all the features, even when the grid template is associated with that type of array. As one simple example, FIG. 5C shows an image 550 having two subarrays 560A and 560B thereon, each of which was deposited with a spotter having a bent pin. As is readily visually observable, the bottommost, rightmost feature 564 in each subarray 560a,560B occupies a position that is distorted from the regular grid formation of the remaining features. In such an instance, grid template 512 cannot be accurately fitted to all features, since it will not be able to overlay and match feature 564 at the same time that it matches the remainder of the features. Therefore, the user in this instance needs to go into grid mode where the system permits manual gridding or editing an exiting grid of an array/subarray by the user interactively, manually locating the nodes of the grid. FIG. 5D shows the results of a manually adjusted grid 562 that may be used to overly and locate the features of subarray 560A.

In this instance, since the error/abnormality is consistently repeated, as having been caused by a systematic error that is repeated in the same way with each deposition of an array/subarray, grid 562 can also be used to overlay and locate the features of subarray 560B and any additional subarrays that are deposited by the same spotter with that bent pin. Rather than force the user to go to grid mode for each of these subarrays and repeat the manual construction of a grid for each of these subarrays, the system allows the user to save grid 562 as another grid template in the database that can then simply be added, populated or applied to each extraction that includes an array/subarray of the type shown in FIG. 5C, thereby greatly reducing the amount of manual input required of the user.

A grid file contains additional information to what is contained in a grid template, and is typically tailored to a specific file or small group of files. For example, a grid file may contain all of the information of the grid template that describes an image array/subarray, and additionally may contain more specific information, such as the location of the origin (e.g., leftmost, uppermost feature in the array/subarray), the rotation and/or skew of the array/subarray or other information which is specific to a particular array/subarray. One way of obtaining this specific information is to overlay a grid template for an image array/subarray and adjust for rotation, skew, accurate alignment of the features, etc., using the techniques described previously. The system then permits this very specific information obtain from manipulating the grid template, to be stored in a grid file, along with the information from the grid template. In this way, future processing of that particular image file can employ the grid file to specifically locate the features of that file with great accuracy.

Typically, a grid file is not stored on the database since it is particular to a user's image file, but is stored locally, such as on the user's hard drive along with the image file, or some other local storage device. A grid file is particularly useful for an image that contains not a systematic error, but an individualized error, such as a scratch, smudge, or some other anomaly that is particular to that file only. A grid file may need to be manually gridded when automatic gridding, skew and rotation compensation fail or are only partly successful. Centroid locations of the features may even be stored in a grid file, so that features may be irregularly spaced but still able to be accurately located by the system using the grid file. Individual spot sizes may also be stored in the grid file.

Not only is the system capable of batch processing image files according to different protocols and/or grid templates, as described above, but the system is also capable of automatically processing multipack images with or without single image files in a batch process. A multipack image is an image resulting from scanning a slide having multiple arrays on the same slide, where each array contains the same design of probes. Typically the arrays on a multipack slide will be hybridized differently, however, so that different results may be achieved on each array, allowing parallel processing of multiple experiments all on the same slide.

The system is adapted to image process an entire slide/image, but post process per hybridization. Thus, a multipack image is initially processed to grid all of the arrays together for location of features. Once features have been located, divisions between the arrays are determined, and each array is processed individually as to post processing (e.g., background subtraction, dye normalization, etc.) to determine the results for each array individually.

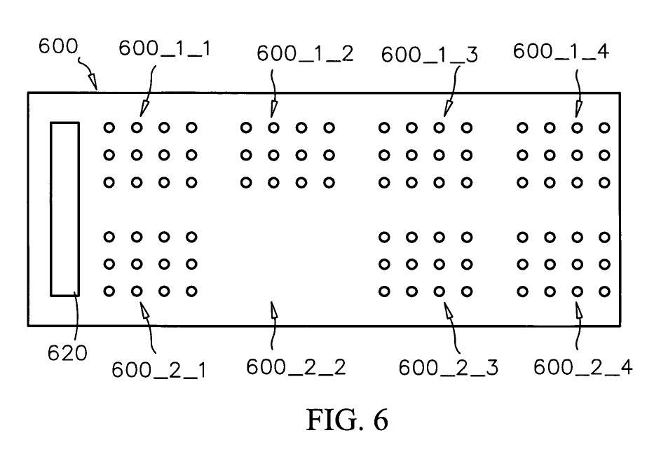

There are distinct advantages to image processing the entire image containing multiple arrays. One advantage is that finding feature location does not have to be repeated multiple time for similar geometries of the multiple arrays contained in the image. Another advantage lies in that, since the geometries of the arrays are similar, there is redundancy provided by the repeating pattern of the array when all are considered together. This may be particularly useful when some features in various arrays are dim or non-existent and would be difficult to locate on the basis of gridding the single array in which the anomalies occur. Even more prominent is the advantage gained in identifying features in an array where no features are readily detectable, by relying on the gridding locations provided by gridding the arrays together. An example of this is schematically shown in FIG. 6 wherein array image 600_2_2 of multipack image 600 represents a “mock hyb” (i.e., array the probes of which have not been hybridized). In such a situation, it is algorithmically more advantageous to find the grid positions of all the individual arrays together rather than one array at a time. Further information regarding algorithmic considerations for locating features can be found in application Ser. No. 10/869,343 filed Jun. 16, 2004 and titled “System and Method of Automated Processing of Multiple Microarray Images” and in application Ser. No. 10,449,175. application Ser. No. 10/869,343 is hereby incorporated by reference herein, in its entirety, by reference thereto, and application Ser. No. 10,449,175 has already been incorporated by reference above. In the disclosure of application Ser. No. 10/869,343, it is not possible to split the image processing and post processing steps of the analysis, and images are cropped to provide eight single array images from and eight pack multi array image. The present system is capable of imaging the eight pack as a single image, as already noted, therefore the user need only save one image file, as opposed to eight.

After the grid is laid and the system has calculated signal statistics (e.g., mean spot signals for the colors, standard deviations for the spot signals for each color, etc.) for each feature, the system moves to post processing. Post processing is done on a per array basis, rather than a per image basis, since each array typically has a different hybridization and may need a different protocol for data analysis. Also, since the hybridizations are separate the user will typically want separate outputs corresponding to the separate arrays. Post processing may include background subtraction processing, outlier rejection processing, dye normalization, and finding/calculating expression ratios. The protocols for image or post processing are typically XML files that contain the parameters of the algorithms to be used in feature extracting an array image.

A typical automatic processing of a multipack image will now be described with regard to an eight pack image, although the system is not limited to automatically processing eight pack multi images, but may also automatically process two pack multi images and other multipack images. Initially, a multipack image, such as image 600, for example is added to a feature extraction project (such as by adding to a feature extract tree 410 or some other visual construct), where a graphical representation for an extraction (e.g., an extraction node or some other representation) is automatically opened to contain the processing information for processing image 600. The system reads the barcode 620 or other identifier and looks up the design file/grid template for image 600. From the design file/grid template, the system learns that image 600 is a multipack image containing eight arrays. A hint may also be contained indicating that the image is a multi-hyb format, so that each of the arrays has a different hybridization. The grid template is also automatically populated into the extraction, in the manner described above, and the protocol may also be automatically populated.

The system then performs a single image processing on the entire image (all eight arrays) together, after which post processing is done individually on each array. Thus, there is one extraction unit corresponding to an eight pack slide and eight outputs of information from post processing for each output format selected (output formats are described in more detail below).

As noted, the design file for the grid template for a multipack image generally includes a hint that indicates that multiple hybridizations are on a slide/image. However, the feature coordinates 110 are only contained for one array on a multipack slide (e.g., the array in the meta-row one, meta-column one position), since the probes on each array are the same. This presents a potential problem for automatic processing by the present system. The problem is presented when a multipack image has been separated/cropped according to the techniques provided in application Ser. No. 10/869,343, which breaks a multipack image down into multiple number of single image files. The problem is presented by the fact that the same identifier/barcode information remains with each image broken out of the multipack image.

Referring back to the previous example where the system automatically processed an eight pack image, assume now that a single image which had been cropped from the eight pack image was added to a Feature Extraction Project 400. Since this single image has the same barcode information as the eight pack image, when the system looks up the grid template, the information contained in the design file of the grid template tells the system that the image to be processed is a multipack image. However, the system also knows the image file as such dimensions are stored in the properties stored with regard to the image.

The system first assumes that the image to be processed is an eight pack image and tries to overlay the pattern stored by the grid template over eight positions where the arrays are expected to be. The dimensions of the attempted overlays are then compared to the dimensions of the image. If at least one of the dimensions of the overlays is larger than the corresponding dimension of the image (i.e., not all of attempted overlays will fit inside the scan dimensions of the image), then it is determined that the image is a single array image, and the grid template is overlaid only once over the image to locate the features in the single array. On the other hand, if the dimensions of the attempted overlays are less than or equal to the dimension of the image, then it is concluded that the image is an eight pack and the system processes to find features using all eight grids.

Looking forward, the dichotomy between image processing on a per slide basis (i.e., proceeding the entire image for features, whether it is a multi array image or a single array image) and post processing and outputting on a per array basis, removes ambiguity as to the unit work piece to be handled. Thus, after adopting this approach, a user will know that all image files, if needed to be further processed, will be representative of an entire slide, and all output files will be representative of a single array each.

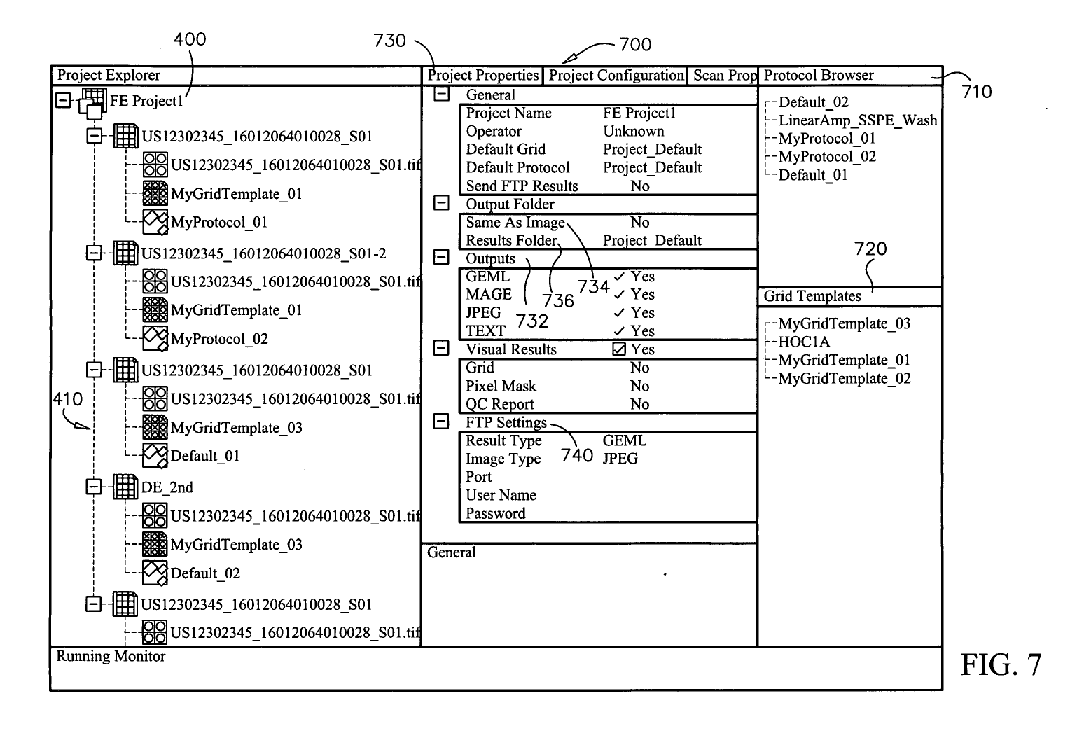

FIG. 7 is a representative screen view 700 that shows feature extraction project 400 (FEProject1) having been populated with extractions so that a batch processing is ready to be run. As shown, when an image is added to the project 400, the project creates an extraction unit corresponding to that image, and the extraction unit is automatically named as the image(TIFF) name unless that extraction name already exists in the same project, which can be observed when comparing the top extraction unit shown with the extraction unit just beneath the top one.

The system automatically populates the grid template and protocol fields of the extractions when possible, in the manners described above. A default protocol is associated with each grid template/design file. As was noted, when the system cannot identify a linked grid template and protocol through the use of a barcode or other identifier associated with the image, then the system automatically applies project level defaults, or the user can select a grid template and/or protocol.

A default grid template and default protocol may be defined at a global level within the software of the system. Each time a user creates a feature extraction project, the same defaults will be applied as project level default grid template and project level default protocol. However, the user of a project may modify the project level defaults for a particular project, thereby overriding the global defaults with regard to that particular feature extraction project. When no other automatic decision can be made with regard to populating a grid template and/or protocol, then the project level defaults are used for these extractions.

Each extraction creates one or more outputs. If an extraction contains a multipack image, then multiple outputs are created for that extraction, one output for each array contained on the image. If the extraction contains a single array image then only one output is created for that extraction. Each output may have one or more files associated with it depending upon the output options selected by the user before running the batch process. Output options 732 may be selected in the Project Properties 730 window, and define various formats for outputted results of the feature extraction processing, including various video formats such as GEML, MAGE-ML, JPEG, etc. TEXT output may also be selected, as well as Visual Results, Grid, and QC Report. The reader is referred to co-pending commonly assigned application Ser. No. 09/775,163 filed Jan. 31, 2001 and titled “Reading Chemical Arrays” (published as U.S. 2002/0102558 on Aug. 1, 2002) and co-pending commonly assigned application Ser. No. 10/798,538 filed Mar. 11, 2004 and titled “Method and System for Microarray Gradient Detection”, for more detailed descriptions of output options. application Ser. No. 09/775,163, application Ser. No. 10/798,538, and U.S. Publication No. 2002/0102558 are hereby incorporated herein, in their entireties, by reference thereto. The QC Report outputs may include, but are not limited to: signal statistics, array uniformity and background measurements, dye normalization factors logratios for replicate inliers with regard to array uniformity; outlier statistics (nonuniformity) spatial gradients based on color/channel, array uniformity for negative controls, logratio accuracy/regression analysis, significance levels for inliers with stated pValue, center of gravity, and sensitivity.

There are at least three options that the system may provide the user as to where output files generated from the extractions will be sent and/or stored. The user may chose to save output files for an extraction in a folder along with the image from which they were generated (e.g., see “Same As Image” option in FIG. 7). A second option is that the user may choose a default output folder or storage location at the project level. A third option is that an output folder or storage location can be individually selected for each extraction in a feature extraction batch project.

Outputs have strict naming conventions that are decided at the software level. In a present embodiment, for a single image (single pack) each file of the output corresponding to that single image has a root name give by “Extraction Name_Protocol” and an extension selected from ,jpg for JPEG, .xml for GEML, MAGE_ML.xml for MAGE, .txt for Tab Text (TEXT), .shp for Visual Results, and _grid.csv for Grid, respectively. Naming conventions for mulitpcak images are similar, but contain suffixes referring to the row and column that the particular array is located in on the slide, similar to what is shown in FIG. 6.

A user interface provided by the system may include a “Running Monitor” window (e.g., see FIG. 7) that displays real time messages regarding the progress of the current feature extraction process being run.

When a project 400 is run, the system may generate a project history file that specifies all the settings used for that project 400, e.g., extraction names, images, grid templates protocols, etc. The project history file may be is automatically saved in the system database or other storage location and is not modifiable. The information stored in history (i.e., in a history file) can be employed by a user to easily track the settings of a previous batch run, and to run the same project again, if desired, or to start a new project with the project settings in a history file and, if necessary, modify them somewhat through user input. The user can view an individual image, protocol or grid template included in a project by mouse clicking or using other well known selection technique on its graphical or textual representation on the user interface.

A typical use of the system by a user includes the user opening a new project from a menu selection within a main window displayed by the system. The system then displays a new blank project 400. The user can name the project with a unique name, select a location that the project meta information will be stored in or select an option to browse for scanned images to include in the project. The user may also drag and drop images from another location onto the project. For each image, the user may select a protocol and grid to be used, either individually, or by a group assignment. Alternatively, for images that are bar coded or have other identification for linking the image to a grid template and protocol, the system may automatically assign the grid template and protocol. These assignments can be overridden by the user. A third option is that the system assigns default grid templates and protocols to those images that do not include a barcode or other identifier to link them to grid templates and protocols. These default grid template and protocol assignments can also be overridden by the user. The user further selects a destination for the results files, which may be stored in the same folder 734 as the image from which they are derived or in a results folder 736 so that all results are contained in one folder. Additionally, the user may input one or more ftp addresses to send results to in the FTP Settings window 740. The batch is then ready to “run” and the user can begin the processing by selecting a run function. The history file for the run will be automatically created and saved in the database.

The order (run sequence) in which extractions are processed by the system is typically a sequential order that runs according to the sequential order that is visually displayed on the user interface. After adding the image files to be processed, or at any time after this prior to running a feature extraction project a user can alter the run sequence from the project setup from the user interface by rearranging the extraction units in a batch, such as by physically rearranging the order in which the extractions appear on the user interface, for example. Running of the feature extraction project is referred to as “run mode”, and “configuration mode” is a mode that is used to set up the feature extraction project, prior to running the project in run mode. At any time while the system is in configuration mode, the user may alter the order of extraction, i.e., change the order in which the extractions will be processed by the system during run mode.

For example, in configuration mode, the user adds image files to the feature extraction project, and may optionally override automatically assigned protocols and/or grid templates, as described above. Default setting may be changed for the entire project, as noted. The sequence order for performing the extractions may be changed. The user may also browse, through the user interface, all protocols that are accessible to the system, such as protocols stored in the system database, as well as grid templates that are useable by the system, for example. The user may also edit any protocol that is not locked so that it cannot be enter. Further, the user may lock a protocol so that it cannot be edited going forward. During run mode, the user can view the results of the extractions as they finish, even before the remainder of the extractions for the project have been completed. Thus, as soon as the first extraction is finished, the user can view the results of that extraction, and so forth.

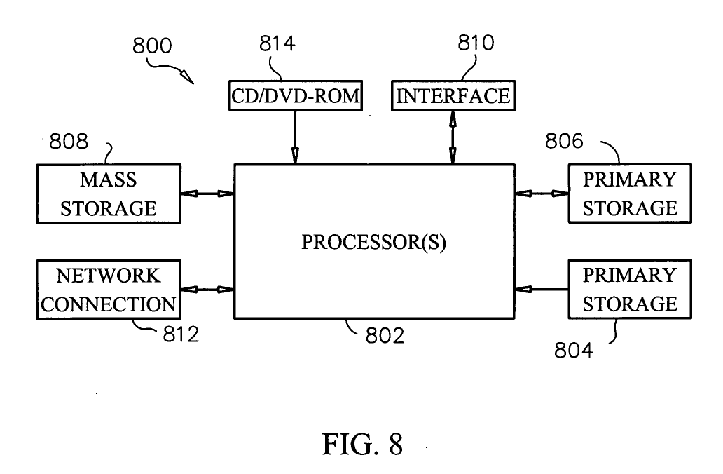

FIG. 8 illustrates a typical computer system that may be used to practice an embodiment of the present invention. The computer system 800 includes any number of processors 802 (also referred to as central processing units, or CPUs) that are coupled to storage devices including primary storage 806 (typically a random access memory, or RAM), primary storage 804 (typically a read only memory, or ROM). As is well known in the art, primary storage 804 acts to transfer data and instructions uni-directionally to the CPU and primary storage 806 is used typically to transfer data and instructions in a bi-directional manner Both of these primary storage devices may include any suitable computer-readable media such as those described above. A mass storage device 808 is also coupled bi-directionally to CPU 802 and provides additional data storage capacity and may include any of the computer-readable media described above. Mass storage device 808 may be used to store programs, data and the like and is typically a secondary storage medium such as a hard disk that is slower than primary storage. It will be appreciated that the information retained within the mass storage device 808, may, in appropriate cases, be incorporated in standard fashion as part of primary storage 806 as virtual memory. A specific mass storage device such as a CD-ROM or DVD-ROM 814 may also pass data uni-directionally to the CPU.

CPU 802 is also coupled to an interface 810 that includes one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 802 optionally may be coupled to a computer or telecommunications network using a network connection as shown generally at 812. With such a network connection, it is contemplated that the CPU might receive information from the network, or might output information to the network in the course of performing the above-described method steps. The above-described devices and materials will be familiar to those of skill in the computer hardware and software arts.

The hardware elements described above may implement the instructions of multiple software modules for performing the operations of this invention. For example, instructions for population of stencils may be stored on mass storage device 808 or 814 and executed on CPU 808 in conjunction with primary memory 806.

In addition, embodiments of the present invention further relate to computer readable media or computer program products that include program instructions and/or data (including data structures) for performing various computer-implemented operations. The media and program instructions may be those specially designed and constructed for the purposes of the present invention, or they may be of the kind well known and available to those having skill in the computer software arts. Examples of computer-readable media include, but are not limited to, magnetic media such as hard disks, floppy disks, and magnetic tape; optical media such as CD-ROM, CDRW, DVD-ROM, or DVD-RW disks; magneto-optical media such as floptical disks; and hardware devices that are specially configured to store and perform program instructions, such as read-only memory devices (ROM) and random access memory (RAM). Examples of program instructions include both machine code, such as produced by a compiler, and files containing higher level code that may be executed by the computer using an interpreter.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A method for automatically feature extracting array images in batch mode, said method comprising the steps of:
loading at least two images to be feature extracted into a batch project; and
automatically and sequentially feature extracting the images in the batch project, wherein at least one of the images is feature extracted based upon a different grid template or protocol than at least one other of the images.

2. The method of claim 1, wherein at least one of the images is a multipack image and at least another of the images is a single array image.

3. The method of claim 1, further comprising automatically assigning a grid template and a protocol to each image.

4. The method of claim 3, wherein the grid template and protocol assignments are linked to an identifier associated with the image.

5. The method of claim 1, wherein any of the images may include an array of any format.

6. The method of claim 1, further comprising generating and storing a history file that specifies images, grid templates and protocols used in processing the batch project.

7. The method of claim 3, wherein a user overrides at least one grid template or protocol assignment and assigns another grid template or protocol to at least one image.

8. The method of claim 3, wherein said automatically assigning comprises automatically assigning a default grid template and a default grid protocol to an image that does not have an identifier associated therewith to link the image to a pre-assigned grid template and protocol.

9. The method of claim 1, wherein said automatic feature extracting includes fitting the grid template onto the image it is assigned to to locate the features, interpreting signals from the features, once they are located, based on the assigned protocol, and outputting results from said interpreting the signals based on the protocol.

10. The method of claim 1, further comprising:
modifying a grid template through user input in grid mode to accommodate a grid template to a systematic error that recurs through a series of array images.

11. The method of claim 1, wherein said automatic feature extracting includes fitting the grid template onto the image it is assigned to to locate the features, said method further comprising storing additional data describing the fitting of the grid template onto the image, along with the grid template, as a grid file.

12. A method comprising forwarding a result obtained from the method of claim 1 to a remote location.

13. A method comprising transmitting data representing a result obtained from the method of claim 1 to a remote location.

14. A method comprising receiving a result obtained from a method of claim 1 from a remote location.

15. A method of automatically feature extracting a single array image having an identifier indicating that it is a multipack, multiple array image, said method comprising:
attempting to overlay an assigned grid template over multiple spaces considered to be occupied by multiple arrays on the image as indicated by a design file upon which the grid template is based;
comparing dimensions of the attempted overlays to dimensions of the image;
determining that the image is a single array image if at least one of the dimensions of the attempted overlays is larger than a corresponding dimension; and
overlaying the grid template only once over the image to locate the features in the single array.

16. The method of claim 15, further comprising fitting the grid template onto the image it is assigned to to locate the features, interpreting signals from the features, once they are located, based on an assigned protocol, and outputting results from said interpreting the signals based on the protocol.

17. A system for feature extracting array images in batch mode, said system comprising:
a user interface with a feature enabling a user to select images to be loaded into a batch project;
means for automatically assigning a grid template to each image loaded into the batch project; and
means for automatically assigning a protocol to each image loaded into the batch project.

18. The system of claim 17, further comprising means for automatically locating features in arrays of said images based upon said grid templates assigned to said images.

19. The system of claim 17, wherein feature extraction of said loaded images is carried out automatically and sequentially.

20. The system of claim 17, further comprising a database storing grid templates and protocols, wherein said automatic assignment of said grid template and protocol is based upon an identifier associated with said image, said identifier being linked with a specific grid template in said database.

21. The system of claim 20, wherein said user interface comprises means for browsing said grid templates and said protocols.

22. The system of claim 20, further comprising means for setting a default protocol and a default grid template to be automatically assigned to an image than lacks said identifier.

23. The system of claim 20, wherein said user interface further comprises a user interactive feature for locking a protocol so that it cannot be further edited.

24. the system of claim 17, wherein said user interface provides an interactive user feature for altering a run order of the extractions according to which the system automatically processes the images.

25. The system of claim 17, wherein the user interface enables a user to override said automatic assignments and substitute a manually inputted assignment of a grid template or protocol.

26. The system of claim 18, further comprising means for determining an amount of adjustment of a grid template required for overlaying the grid template over an image for said automatic location of features.

27. The system of claim 26, further comprising means for warning a user that the grid template is a bad fit with the image when a predetermined percentage of spots on the grid template were moved by greater than or equal to a predetermined distance during said automatic location of features.

28. A computer readable medium carrying one or more sequences of instructions for feature extracting array images in batch mode, wherein execution of one or more sequences of instructions by one or more processors causes the one or more processors to perform the steps of:
loading at least two images to be feature extracted into a batch project; and
automatically and sequentially feature extracting the images in the batch project, wherein at least one of the images is feature extracted based upon a different grid template or protocol than at least one other of the images.

29. The computer readable medium of claim 28, wherein said execution of one or more sequences of instructions causes the one or more processors to perform the further steps, with respect to each image of: fitting the grid template onto the image it is assigned to to locate the features, interpreting signals from the features, once they are located, based on the assigned protocol, and outputting results from said interpreting the signals based on the protocol.
